(19) United States
(12) Patent Application Publication
(10) Pub. No.: US 2010/0074971 A1
(43) Pub. Date: Mar. 25, 2010

(54) DOUBLE PREPARATION TYPE ANTIOXIDANT COMPOSITION AND ANTIOXIDATION PRODUCT COMPRISING THE SAME

(75) Inventors: Kyoko Hirano, Kanagawa (JP); Hiroyasu Kumamoto, Kanagawa (JP); Takashi Kawano, Kanagawa (JP)

Correspondence Address: FITZPATRICK CELLA HARPER & SCINTO, 1290 Avenue of the Americas, NEW YORK, NY, 10104-3800, US

(73) Assignee: TAKASAGO INTERNATIONAL CORPORATION, Ohta-ku, Tokyo (JP)

(21) Appl. No.: 12/517,133

(22) PCT Filed: Oct. 30, 2007
(86) PCT No.: PCT/JP2007/071152

§ 371 Date: Jun. 1, 2009

(30) Foreign Application Priority Data
Dec. 22, 2006 (JP) 2006-346658

(51) Int. Cl. A61K 33/04; A61K 33/00

(52) U.S. Cl. 424/709; 424/717

(57) ABSTRACT

It is to provide an antioxidant excellent in antioxidative potential and having high safety is provided. Also, it is to provide an antioxidant in which problems of unpleasant smell and taste are avoided and its preparation method is not so complex.

Further, it is to provide an antioxidant which can exert its effect by a simple operation when exertion of the antioxidative potential is desired. It relates to a double preparation type antioxidant, which comprises a mixture A comprising a polyphenol and a mixture B comprising an alkali agent. In particular, two antioxidant component-storing containers are mutually integrally molded and combined, and a mixture A comprising a polyphenol is stored in one of the antioxidant component-storing containers and a mixture B comprising an alkali agent is stored in the other antioxidant component storing container.

### TECHNICAL FIELD

This invention relates to a double preparation type antioxidant, and more illustratively, it relates to a double preparation type antioxidant which comprises a mixture A comprising a polyphenol and a mixture B comprising an alkali agent.

### BACKGROUND OF THE INVENTION

It is everyone's desire to keep the skin healthy and beautiful conditions. Actually, many attempts have been made for keeping the skin having flexibility, smoothness, softness, freshness, transparent touch and the like. As one of such attempts, there is a method to use an antioxidant. That is, it is an idea of avoiding aging of the skin by making use of the antioxidant, based on the assumption that superoxide anion, hydroxy radical, active oxygen and the like which are generated by intracellular actions become the cause of aging of the skin.

For example, dibutylhydroxytoluene (BHT), butylhydroxyanisole (BHA) and the like synthetic antioxidants which have excellent antioxidative potential have been used. However, though BHT, BHA and the like antioxidants are effective from the viewpoint of their effects, various problems have been pointed out on these compounds in terms of their safety. On the contrary, ascorbic acid, tocopherol and the like natural antioxidants, and extracts from bamboos, extracts from fermented beans (Patent References 1 and 2) and the like natural antioxidants do not cause the aforementioned problem of the synthetic antioxidants in terms of their safety, but ascorbic acid poses problems such as extreme difficulty in dissolving in fat-soluble substances and tocopherol poses problems such as the presence of peculiar unpleasant smell and taste. In addition, the plant extracts pose problems such as their complex preparation methods.

On the other hand, it is well known that the hair becomes fragile and undergoes aging by the action of environmental factors, particularly light. Since the natural light destroys certain species of an amino acid in the hair and thereby spoils hair fibers, the hair loses gloss and becomes chafed and fragile. In addition, the light exerts an influence upon the natural color of hair or stained hair, so that the color of hair gradually becomes thin or changes into an undesirable hue. The use of an antioxidant or the like for the purpose of protecting hair from its deterioration by a light and the like is already known, but it can be said that the problems in using this antioxidant are also the same as the above-mentioned case of the skin.

### Patent Reference 1: JP-A-2006-116433

### Patent Reference 2: JP-A-2006-70146

### DISCLOSURE OF THE INVENTION

### Problems That the Invention is to Solve

Accordingly, an object of the invention is to provide an antioxidant which is excellent in antioxidative potential and also has high safety. Also, it is to provide an antioxidant in which the problems of malodor and off-taste can be avoided and its preparation method is not so complex. In addition, it is further to provide an antioxidant which can exert its effect by a simple operation when exertion of the antioxidative potential is desired.

### Means for Solving the Problems

In the midst of the intensive studies for solving the above-mentioned problems, the present inventors have unexpectedly obtained information that excellent antioxidation effect can be shown when a mixture containing a polyphenol and a mixture containing an alkali agent are separated in two in advance and then mixed in response to the necessity. By further continuing the studies based on this information, the invention has been accomplished at last.

That is, the invention relates to the following.

1. A double preparation type antioxidant, which comprises a mixture A comprising a polyphenol and a mixture B comprising an alkali agent.

2. The double preparation type antioxidant according to item 1, wherein the mixture B further comprises an aqueous solvent.

3. The double preparation type antioxidant according to item 1 or 2, wherein an amount of the polyphenol contained in the mixture A is from 10⁻⁷ to 10% by mass.

4. The double preparation type antioxidant according to any one of items 1 to 3, wherein pH of a mixture of the mixture A and mixture B is from 7 to 11.

5. A double preparation type antioxidation product, which comprises two antioxidant component-storing containers wherein at least parts of the two antioxidant component-storing containers are mutually integrally molded and combined,

wherein a mixture A comprising a polyphenol is stored in one of the antioxidant component-storing containers, and a mixture B comprising an alkali agent is stored in the other antioxidant component-storing container.

6. A double preparation type antioxidation product, which comprises two antioxidant component-storing containers wherein at least parts of the two antioxidant component-storing containers are mutually integrally molded and combined,

wherein a mixture A comprising a polyphenol is stored in one of the antioxidant component-storing containers, and a mixture B comprising an alkali agent is stored in the other antioxidant component-storing container,

which further comprises a structure in which the mixture A flowing out of an outlet of the mixture A and the mixture B flowing out of an outlet of the mixture B are mutually mixed at around the respective outlets thereof.

7. A double preparation type fragrance or cosmetic or double preparation type oral product, which comprises an agent I comprising the mixture A according to item 1 and an agent II comprising the mixture B according to item 1.

### ADVANTAGE OF THE INVENTION

The double preparation type antioxidant of the invention is an antioxidant which is excellent in antioxidative potential and also has high safety. Also, since the component comprising the polyphenol or the like antioxidant is stably maintained, excellent antioxidative potential can be exerted even after a prolonged period of preservation. What is more, under a situation of wanting exertion of its antioxidative potential, antioxidation potential is exerted within a short period of time by a simple operation. In addition, this is an antioxidant in which the problems of malodor and off-taste were avoided. This is a markedly practical invention, because influences of an active oxygen and/or a free radical and the like can be avoided or reduced by the use of the double preparation type antioxidant of the invention.

### BEST MODE FOR CARRYING OUT THE INVENTION

The following describes the invention in detail.

The polyphenol to be used in the double preparation type antioxidant of the invention is described.

The polyphenol to be used in the invention is not particularly limited so far as it is a polyphenol capable of attaining the intended purpose.

Illustratively, the polyphenol to be used in the invention means a compound in which two or two or more hydroxyl groups on one identical benzene ring are substituted with hydrogen atoms, and glycosides thereof are also included as the polyphenol.

Among them, a polyphenol having a hydroquinone and a polyphenol having an o-diphenol structure are preferred. In this connection, the o-diphenol structure means such a structure that hydroxyl groups are directly substituted on the benzene ring and the hydroxyl groups are adjacent to each other.

Illustrative examples of the polyphenol include apigenin, apigenin glycosides, acacetin, isorhamnetin, isorhamnetin glycosides, isoquercitrin, epicatechin, epicatechin gallate, epigallocatechin, epigallocatechin gallate, aesculetin, ethyl protocatechuate salt, ellagic acid, catechol, gamma acid, catechin, gardenin, gallocatechin, caffeic acid, caffeic acid esters, chlorogenic acid, kaempferol, kaempferol glycosides, quercetin, quercetin glycosides, quercetagenin, genistin, genistin glycosides, gossypectin, gossypectin glycosides, gossypol, 4-dihydroxyanthraquinone, 1,4-dihydroxynaphthalene, cyanidin, cyanidin glycosides, sinensetin, diosmetin, diosmetin glycosides, 3,4′-diphenyldiol, sinapic acid, stearyl-β-(3,5-di-t-butyl-4-hydroxyphenyl) propionate, spinacene, tangeritin, taxifolin, tannic acid, daphnetin, tyrosine, delphinidin, delphinidin glycosides, theaflavine, theaflavine monogallate, theaflavine bisgallate, tricetinidin, dopa, dopamine, naringenin, naringin, nordihydroguairetic acid, noradrenaline, hydroquinone, vanillin, patchouletin, herbacetin, vanillyl alcohol, vanitroppe, vanillin propylene glycol acetal, vanillic acid, bis(4-hydroxyphenyl)sulfonic acid, bisphenol A, pyrocatechol, vitexin, 4,4′-biphenyldiol, 4-t-butylcatechol, 2-t-butylhydroquinone, protocatechuic acid, phloroglucinol, phenolic resins, procyanidin, prodelphinidin, phloretin, phloretin glycosides, fisetin, folin, fervasetin, fraxetin, phloridzin, paeonidin, paeonidin glycosides, pelargonidin, pelargonidin glycosides, petunidin, petunidin glycosides, hesperetin, hesperidin, gallic acid, gallic acid esters (lauryl gallate, propyl gallate, butyl gallate), manjiferin, malvidin, malvidin glycosides, myricetin, myricetin glycosides, 2,2′-methylenebis(4-methyl-6-t-butylphenol), 2,2% methylenebis(4-ethyl-6-t-butylphenol), 2,2′-methylenebis(4-methyl-6-t-butylphenol), 2,2′-methylenebis(4-ethyl-6-t-butylphenol), methyl atrarate, 4-methylcatechol, 5-methylcatechol, 4-methoxycatechol, 5-methoxycatechol, methylcatechol-4-carboxylic acid, 2-methylresorcinol, 5-methylresorcinol, morin, lignin, limocitrin, limocitrin glycosides, limocitrol, luteolin, luteorin glycosides, luteolinidin, luteolinidin glycosides, rutin, resorcin, resveratrol, resorcinol, leukocyanidin, leukodelphinidin, and the like.

Among these polyphenols, particularly preferred are quercetin, epicatechin, epigallocatechin and the like flavonoids and glycosides thereof, gallic acid, gallic acid esters, chlorogenic acid, caffeic acid, caffeic acid esters, tannic acid, pyrocatechol, nordihydroguairetic acid, L-dopa, 4-methylcatechol, 5-methylcatechol, 4-methoxycatechol, 5-methoxycatechol and the like polyphenols having an o-diphenol structure, and hydroquinone.

The polyphenols may be used each alone or used by mixing two or more kinds thereof. Also, the aforementioned polyphenols can be prepared by known methods, but commercially available products may be purchased. Moreover, they may be prepared by synthesis. In addition, polyphenol fractions prepared from plants can also be used.

In the invention, instead of the polyphenol, a polyphenol-containing plant extract can also be used. As said plant extract, one prepared by a known method may be used or a commercially available product may be used.

Examples of the plant extract include plant extracts obtained through an extraction treatment by any usual methods from, for example, aloe, anise seeds, elder, eleutherococcus, psyllium, orange flower, allspice, oregano, valerian, chamomile, capsicum pepper, cardamom, cassia, garlic, caraway seeds, clove, cumin seeds, kola, coriander seeds, Rhus javanica, saffron, zanthoxylum, juniper berry, cinnamon, ginger, star anise, St. Johns wart, celery seed, savory, sesame, pieplant, tarragon, turmeric, thistle, dill seeds, nutmeg, nettle, hibiscus, hamamelis, birch, basil, bitter orange, fennel, primrose, fenugreek, verbena, bay laurel, hop, boldo, horseradish, poppy seed, gallnut, marigold, marrow, majoram, mustard, Millefeuille, mint leaves, melissa, mace, lindane, gentian, rosehip, rosemary, Rosmarinus officinalis L., sunflower seeds, grape pericarp, apple, carrot leaves, banana, strawberry, apricot, peach, plum, pineapple, Nashi pear, persimmon, cherry, papaya, mango, avocado, melon, loquat, fig, kiwi, prune, blueberry, black berry, raspberry, cranberry, coffee beans, cacao beans, grape seeds, grape fruits seeds, pecan nut, cashew nut, chestnut, coconut, peanut, walnut, green tea leaves, black tea leaves, oolong tea leaves, tobacco, perilla leaves, garden thyme, sage, lavender, spearmint, peppermint, spotted thistle, hyssop, sweet basil, marigold, dandelion, artichoke, Matricaria chamomile, Agrimonia pilosa var. japonica, licorice, anise, yarrow, eucalyptus, wormwood, balm, Angelica pubescens, fenugreek, Capsicum annuum var. angulosum, fennel, red pepper, coriander seeds, caraway seeds, fennel seeds, ginger, horseradish, Origanum majorana, Origanum valgare, mustard, parsley, pepper, savory, tarragon, queen lily, wasabi, dill seeds, citrus fruits and the like. These plant extracts may be used alone or in combination of two or more plant extracts.

In this connection, the polyphenol compound and the polyphenol-containing plant extract may be used in combination.

Next, it is desirable to contain an acid agent in the mixture A according to the invention. The acid agent to be used in the invention is not particularly limited so fat as it is an acid agent capable of attaining the intended purpose.

As illustrative examples of the acid agent, for example, one or two or more kinds selected from the group consisting of glycolic acid, glyceric acid, tartaric acid, citric acid, ascorbic acid, malic acid, succinic acid, fumaric acid, adipic acid, an acidic amino acid, potassium chloride-hydrochloride buffer, potassium hydrogen phthalate-sodium hydride buffer, sodium citrate-sodium hydroxide and the like, can be exemplified. As the acidic amino acid, for example, aspartic acid, glutamic acid and the like can be exemplified.

The mixture A according to the invention can further contain a solvent such as an aqueous solvent and can thereby be made into a liquid mixture. As the suitable solvent, for example, methanol, ethanol, isopropanol, ethylene glycol, propylene glycol, diethylene glycol, dipropylene glycol and glycerin can be exemplified. In addition, according to the invention, purified water, ion exchange water, pure water and the like can also be regarded as the aqueous solvent.

The mixture A also includes a mixture having an extremely high viscosity. For example, it also includes a mixture which is stored in a resin-made tubular container and from which the contents are taken out by pressing the container as occasion demands.

In the mixture A according to the invention, in order to efficiently exert the antioxidation effect, the content of the polyphenol is set to preferably from 10⁻⁷ to 20% by mass, more preferably from 10⁻⁶ to 10% by mass, particularly preferably from 10⁻⁶ to 5% by mass, based on the total amount of the mixture A, though it varies depending on the polyphenol to be used. Also, though it varies depending on the acid agent to be used, the content of the acid agent is set to preferably from 0.001 to 20% by mass, more preferably from 0.01 to 10% by mass, based on the total amount of said mixture A. This is because the antioxidation effect is not sufficient when the polyphenol or acid agent is less than the aforementioned range and when it is more than the aforementioned range, the case is not desirable in view of handling properties and the like.

In this connection, it is desirable to set an acidity of the mixture A to pH of from 1.0 to 6.0, more desirably pH of from 2.0 to 6.0, though it varies depending on the polyphenol and acid agent to be used. This is because the polyphenol can be stably present within this range due to efficient antioxidation action.

Next, the alkali agent to be contained in the mixture B according to the invention is described. The alkali agent to be used in the invention is not particularly limited so far as it is an alkali agent capable of attaining the intended purpose. As illustrative examples of the alkali agent, for example, at least one kind selected from sodium carbonate, sodium hydrogen carbonate, potassium carbonate, potassium hydrogen carbonate, disodium hydrogen phosphate, dipotassium hydrogen phosphate, sodium citrate, potassium citrate, sodium hydroxide, potassium hydroxide, sodium carbonate-sodium hydrogen carbonate buffer, disodium hydrogen phosphate-sodium hydroxide buffer and the like can be exemplified.

The mixture B according to the invention may contain an aqueous solvent. As the aqueous solvent, it is the same as the case of the above-mentioned mixture A. The mixture B also includes a mixture having an extremely high viscosity similar to the case of the mixture A.

With regard to the mixture B according to the invention, the content of the alkali agent varies depending on the alkali agent to be used, the shape of the double preparation type antioxidant and the like, so that it cannot be categorically defined, but in daringly saying, when the mixture B is liquid, it is desirable to set it to from 0.001 to 20% by mass based on the amount of said mixture B, and when the mixture B is solid, it is desirable to set it to from 20 to 100% by mass based on the amount of said mixture B.

In this connection, a basicity of the mixture B varies depending on the alkali agent to be used but is set to preferably pH of from 7.5 to 13.0, more preferably pH of from 8.0 to 12.5, particularly preferably pH of from 8.5 to 12.0. This is because it can exert the antioxidation action efficiently when within this range.

According to the invention, a fragrance suitable for individual components may be blended in advance into the mixture A and mixture B from the viewpoint of odorous diffusivity and the like.

According to the invention, the fragrance to be blended into respective components is not particularly limited so far as it is an organic compound generally vaporized into the air at room temperature and is an organic compound effective as a fragrance, but it is desirable that the fragrance to be blended into the mixture A is a fragrance which is stable to the polyphenol and the like components contained in the mixture A. This is because when a fragrance unstable to the acid agent and polyphenol is blended into the mixture A, with the lapse of time, there may easily occur a disfigurement of appearance of the mixture B through a degradation of the fragrance, change in color, and precipitation, and destruction of harmony of aroma.

Also, it is desirable that the fragrance to be blended into the mixture B is a fragrance which is stable to the alkali agent. This is because when a fragrance unstable to the alkali agent is blended into the mixture B, with the lapse of time, there may easily occur a disfigurement of appearance of the mixture B through a degradation of the fragrance, change in color, and precipitation, and destruction of harmony of aroma. Accordingly, it is desirable to select a fragrance stable to individual components as individual fragrances to be blended into the mixture A and mixture B.

Into the mixture A according to the invention, it is desirable to blend at least one or more kinds selected from an alcohol-type aromachemical, an ester-type aromachemical, a hydrocarbon-type aromachemical, a lactone-type aromachemical, an ether-type aromachemical and a natural origin aromatic material which are fragrances stable to the polyphenol and acid agent. The amount of these fragrances to be blended is not particularly limited, but it is desirable to set it to, for example, from 30 to 100% by mass, more desirably to set to from 30 to 80% by mass, based on all the fragrances to be blended into the mixture A.

Into the mixture B according to the invention, it is desirable to blend at least one or more kinds selected from an alcohol-type aromachemical, an aldehyde-type aromachemical, a ketone-type aromachemical, an ether-type aromachemical and a natural origin aromatic material which are fragrances stable to the alkali agent. The amount of these fragrances to be blended is not particularly limited, but it is desirable to set it to, for example, from 30 to 100% by mass, more desirably to set to from 30 to 80% by mass, based on all the fragrances to be blended into the mixture B.

In addition, by incorporating the aforementioned fragrances into the mixture A and/or mixture B, it becomes possible to maintain a stable balanced aroma, respectively. In this connection, the fragrance in the antioxidant is useful also in masking a malodor by the aroma of the fragrance, and in that case, it is desirable to be masked by a good-feeling and comfortable aroma to the user.

According to the invention, the fragrance to be used in the mixture A is a so-called fragrance compound, and it is desirable that the fragrance has a well-balanced odor quality and a good-feeling and comfortable aroma to the user and is a fragrance for masking, and the mixture B is also the same the above so that it is desirable that the fragrance to be used in the mixture A has a well-balanced aroma and a good-feeling and comfortable aroma to the user and is a fragrance for masking.

Illustrative examples of the fragrance to be used in the invention are as follows.

As the alcohol-type aromachemical, there may be mentioned citronellol, geraniol, linarol, borneol, menthol, nerol, cis-3-hexenol, terpineol, tetrahydrolinarol, β-phenylethyl alcohol, cinnamic alcohol, anise alcohol, dimethylbenzyl carbinol, phenoxyethyl alcohol, sabinene hydrate, bacdanol and santalol.

As the natural origin aromatic material, there may be mentioned peppermint oil, mentha oil, rosemary oil, eucalyptus oil, tea tree oil, orange oil, lavender oil and geranium oil.

As the ether-type aromachemical, there may be mentioned galaxolide, rose oxide, linarol oxide, cineol, cedramber, dibenzyl ether, ambroxan, diphenyl oxide, β-naphthol methyl ether and β-naphthol ethyl ether.

Also, as the aldehyde-type aromachemical, there may be mentioned cis-3-hexenal, octanal, nonanal, decanal, citroneral, perillaldehyde, α-amyl cinnamic aldehyde, α-hexyl cinnamic aldehyde, helional, lilinal, vanillin and triplal.

As the ketone-type aromachemical, there may be mentioned camphor, menthone, ionone, methylionone, tonalid, muscone, cyclopentadecane, cashmeran, damascene, damascenone and cis-jasmone.

As the ester-type aromachemical, there may be mentioned citronellyl acetate, geranyl acetate, neryl acetate, benzyl acetate, terpinyl acetate, ethyl acetate, propyl acetate, butyl acetate, amyl acetate, bornyl acetate, menthyl acetate, p-tert-butylcyclohexyl acetate, o-tert-butylcyclohexyl acetate, phenylethyl acetate and styralyl acetate.

As the hydrocarbon-type aromachemical, there may be mentioned limonene, pinene, diphenylmethane, myrcene and sabinene.

As the lactone-type aromachemical, there may be mentioned coumarin, γ-undecalactone, cyclopentadecanolide and γ-nonalactone.

According to the invention, the amount of the fragrances to be blended into the mixture A or mixture B varies depending on the kinds and amounts of the chemical substances blended in the mixture A or mixture B and thus is not categorically defined, but in order to provide preferable aroma, it is preferably 0.001% by weight or more and is preferably less than 60% by weight based on the total amount of the mixture A or mixture B. This is because periodical stability of aroma and harmony of aroma are not sufficient when it is less than 0.001%, and well-balanced preferable aroma cannot be added when it is 60% by weight or more.

According to the invention, the other fragrances and solvents for fragrances can be further incorporated in such a range that the advantages of the invention are not impaired. The other usable fragrances are mentioned in various documents, for example, “Perfume and Flavor Chemicals”, Vol. I and II, Steffen Arctander, Allured Pub. Co. (1994) and the like.

In the following, for example, esters, alcohols, aldehydes, ketones, acetals, phenols, ethers, lactones, furans, hydrocarbons and acids are mentioned as typical examples of the other usable fragrance, though not limited thereto so far as these are within such a range that the advantages of the invention are not impaired.

As the esters, for example, there may be mentioned acrylate esters (methyl, ethyl and the like), acetoacetate esters (methyl, ethyl and the like), anisate esters (methyl, ethyl and the like), benzoate esters (allyl, isoamyl, ethyl, geranyl, linalyl, phenylethyl, hexyl, cis-3-hexenyl, benzyl, methyl and the like), anthranilate esters (cinnamyl, cis-3-hexenyl, methyl, ethyl, linalyl, isobutyl and the like), N-methylanthranilate esters (methyl, ethyl and the like), isovalerate esters (amyl, allyl, isoamyl, isobutyl, isopropyl, ethyl, octyl, geranyl, cyclohexyl, citronellyl, terpenyl, linalyl, cinnamyl, phenylethyl, butyl, propyl, hexyl, benzyl, methyl, rhodinyl and the like), isobutyrate esters (isoamyl, geranyl, citronellyl, terpenyl, cinnamyl, octyl, neryl, phenylethyl, phenylpropyl, phenoxyethyl, butyl, propyl, isopropyl, hexyl, benzyl, methyl, ethyl, linalyl, rhodinyl and the like), undecylenate esters (allyl, isoamyl, butyl, ethyl, methyl and the like), octanoate esters (allyl, isoamyl, ethyl, octyl, hexyl, butyl, methyl, linalyl and the like), octenoate esters (methyl, ethyl and the like), octynecarboxylate esters (methyl, ethyl and the like), caproate esters (allyl, amyl, isoamyl, methyl, ethyl, isobutyl, propyl, hexyl, cis-3-hexenyl, trans-2-hexenyl, linalyl, geranyl, cyclohexyl and the like), hexenoate esters (methyl, ethyl and the like), valerate esters (amyl, isopropyl, isobutyl, ethyl, cis-3-hexenyl, trans-2-hexenyl, cinnamyl, phenylethyl, methyl and the like), formate esters (anisyl, isoamyl, isopropyl, ethyl, octyl, geranyl, citronellyl, cinnamyl, cyclohexyl, terpinyl, phenylethyl, butyl, propyl, hexyl, cis-3-hexenyl, benzyl, linalyl, rhodinyl and the like), crotonate esters (isobutyl, ethyl, cyclohexyl and the like), cinnamate esters (allyl, ethyl, methyl, isopropyl, propyl, 3-phenylpropyl, benzyl, cyclohexyl, methyl and the like), succinate esters (monomenthyl, diethyl, dimethyl and the like), acetate esters (anisyl, amyl, α-amylcinnamyl, isoamyl, isobutyl, isopropyl, isobornyl, isoeugenyl, eugenyl, 2-ethylbutyl, ethyl, 3-octyl, p-cresyl, o-cresyl, geranyl, α- or β-santalyl, cyclohexyl, cycloneryl, dihydrocuminyl, dimethylbenzylcarbinyl, cinnamyl, styrallyl, decyl, dodecyl, terpinyl, guanyl, neryl, nonyl, phenylethyl, phenylpropyl, butyl, furfuryl, propyl, hexyl, cis-3-hexenyl, trans-2-hexenyl, cis-3-nonenyl, cis-6-nonenyl, cis-3,cis-6-nonadienyl, 3-methyl-2-butenyl, heptyl, benzyl, bornyl, myrcenyl, dihydromyrcenyl, myrtenyl, methyl, 2-methylbutyl, menthyl, linalyl, rhodinyl and the like), salicylate esters (allyl, isoamyl, phenyl, phenylethyl, benzyl, ethyl, methyl and the like), cyclohexylalkanoate esters (ethyl cyclohexylacetate, allyl cyclohexylpropionate, allyl cyclohexylbutyrate, allyl cyclohexylhexanoate, allyl cyclohexyldecanoate, allyl cyclohexylvalerate and the like), stearate esters (ethyl, propyl, butyl and the like), sebacate esters (diethyl, dimethyl and the like), decanoate esters (isoamyl, ethyl, butyl, methyl and the like), dodecanoate esters (isoamyl, ethyl, butyl and the like), lactate esters (isoamyl, ethyl, butyl and the like), nonanoate esters (ethyl, phenylethyl, methyl and the like), noneno ate esters (allyl, ethyl, methyl and the like), hydroxyhexanoate esters (ethyl, methyl and the like), phenylacetate esters (isoamyl, isobutyl, ethyl, geranyl, citronellyl, cis-3-hexenyl, methyl and the like), phenoxyacetate esters (allyl, ethyl, methyl and the like), furancarboxylate esters (ethyl furancarboxylate, methyl furancarboxylate, hexyl furancarboxylate, isobutyl furanpropionate and the like), propionate esters (anisyl, allyl, ethyl, amyl, isoamyl, propyl, butyl, isobutyl, isopropyl, benzyl, geranyl, cyclohexyl, citronellyl, cinnamyl, tetrahydrofurfuryl, tricyclodecenyl, heptyl, bornyl, methyl, menthyl, linalyl, terpinyl, α-methylpropionyl, β-methylpropionyl and the like), heptanoate esters (allyl, ethyl, octyl, propyl, methyl and the like), heptynecarboxylate esters (allyl, ethyl, propyl, methyl and the like), myristate esters (isopropyl, ethyl, methyl and the like), phenylglycidate esters (ethyl phenylglycidate, ethyl 3-methylphenylglycidate, ethyl p-methyl-β-phenylglycidate and the like), 2-methylbutyrate esters (methyl, ethyl, octyl, phenylethyl, butyl, hexyl, benzyl and the like), 3-methylbutyrate esters (methyl, ethyl and the like), butyrate esters (anisyl, amyl, allyl, isoamyl, methyl, ethyl, propyl, octyl, guanyl, linalyl, geranyl, cyclohexyl, citronellyl, cinnamyl, neryl, terpenyl, phenylpropyl, β-phenylethyl, butyl, hexyl, cis-3-hexenyl, trans-2-hexenyl, benzyl, rhodinyl and the like), hydroxybutyrate esters (methyl, ethyl or menthyl 3-hydroxybutyrate and the like) and the like.

As the alcohols, for example, there may be mentioned aliphatic alcohols (isoamyl alcohol, 2-ethylhexanol, 1-octanol, 3-octanol, 1-octen-3-ol, 1-decanol, 1-dodecanol, 2,6-nonadienol, nonanol, 2-nonanol, cis-6-nonenol, trans-2, cis-6-nonadienol, cis-3, cis-6-nonadienol, butanol, hexanol, cis-3-hexenol, trans-2-hexenol, 1-undecanol, heptanol, 2-heptanol, 3-methyl-1-pentanol and the like), terpene alcohols (borneol, isoborneol, carveol, geraniol, α- or β-santalol, citronellol, 4-thujanol, terpineol, 4-terpineol, nerol, myrcenol, myrtenol, dihydromyrcenol, tetrahydromyrcenol, nerolidol, hydroxycitronerol, farnesol, perilla alcohol, rhodinol, linalool and the like), aromatic alcohols (anise alcohol, α-amylcinnamic alcohol, isopropylbenzylcarbinol, carvacrol, cuminic alcohol, dimethylbenzylcarbinol, cinnamic alcohol, phenylallyl alcohol, phenylethylcarbinol, β-phenylethyl alcohol, 3-phenylpropyl alcohol, benzyl alcohol and the like) and the like.

As the aldehydes, for example, there may be mentioned aliphatic aldehydes (acetaldehyde, octanal, nonanal, decanal, undecanal, 2,6-dimethyl-5-heptanal, 3,5,5-trimethylhexanal, cis-3,cis-6-nonadienal, trans-2,cis-6-nonadienal, valeraldehyde, propanal, isopropanal, hexanal, trans-2-hexenal, cis-3-hexenal, 2-pentenal, dodecanal, tetradecanal, trans-4-decenal, trans-2-tridecenal, trans-2-dodecenal, trans-2-undecenal, 2,4-hexadienal, cis-6-nonenal, trans-2-nonenal, 2-methylbutanal and the like), aromatic aldehydes (anisaldehyde, α-amylcinnamic aldehyde, α-methylcinnamic aldehyde, cyclamen aldehyde, p-isopropylphenylacetaldehyde, ethylvanillin, cuminaldehyde, salicylaldehyde, cinnamic aldehyde, o-, m- or p-tolylaldehyde, vanillin, piperonal, phenylacetaldehyde, heliotropin, benzaldehyde, 4-methyl-2-phenyl-2-pentenal, p-methoxycinnamic aldehyde, p-methoxybenzaldehyde and the like), terpene aldehydes (geranial, citral, citronellal, α-sinensal, β-sinensal, perillaldehyde, hydroxycitronellal, tetrahydrocitral, myrtenal, cyclocitral, isocyclocitral, citronellyloxyacetaldehyde, neral, α-methylenecitronellal, myrac aldehyde, vernaldehyde, safranal and the like) and the like.

As the ketones, for example, there may be mentioned cyclic ketones (1-acetyl-3,3-dimethyl-1-cyclohexene, cis-jasmone, α-, β- or γ-irone, ethylmaltol, cyclotene, dihydronootkatone, 3,4-dimethyl-1,2-cyclopentadione, sotolone, α-, β-, γ- or δ-damascone, α-, β- or γ-damascenone, nootkatone, 2-sec-butylcyclohexanone, maltol, α-, β- or γ-ionone, α-, β- or γ-methylionone, α-, β- or γ-isomethylionone, furaneol, camphor and the like), aromatic ketones (acetonaphthone, acetophenone, anisylideneacetone, raspberry ketone, p-methylacetophenone, anisylacetone, p-methoxyacetophenone and the like), linear ketones (diacetyl, 2-nonanone, diacetyl, 2-heptanone, 2,3-heptanedione, 2-pentanone, methyl amyl ketone, methyl nonyl ketone, n-methyl naphthyl ketone, methylheptanone, 3-heptanone, 4-heptanone, 3-octanone, 2,3-hexanedione, 2-undecanone, dimethyloctenone, 6-methyl-5-heptyn-3-one and the like) and the like.

As the acetals, for example, there may be mentioned acetaldehyde diethyl acetal, acetaldehyde diamyl acetal, acetaldehyde dihexyl acetal, acetaldehyde propylene glycol acetal, acetaldehyde ethyl cis-3-hexenyl acetal, benzaldehyde glycerin acetal, benzaldehyde propylene glycol acetal, citral dimethyl acetal, citral diethyl acetal, citral propylene glycol acetal, citral ethylene glycol acetal, phenylacetaldehyde dimethyl acetal, citronellyl methyl acetal, acetaldehyde phenylethyl propyl acetal, hexanal dimethyl acetal, hexanal dihexyl acetal, hexanal propylene glycol acetal, trans-2-hexanal diethyl acetal, trans-2-hexanal propylene glycol acetal, cis-3-hexanal diethyl acetal, heptanal diethyl acetal, heptanal ethylene glycol acetal, octanal dimethyl acetal, nonanal dimethyl acetal, decanal dimethyl acetal, decanal diethyl acetal, 2-methylundecanal dimethyl acetal, citronellal dimethyl acetal, ambersage (manufactured by Givaudan), ethyl acetoacetate ethylene glycol acetal, 2-phenylpropanal dimethyl acetal and the like.

As the phenols, for example, there may be mentioned eugenol, isoeugenol, 2-methoxy-4-vinylphenol, thymol, carvacrol, guaiacol, chavicol and the like.

As the ethers, for example, there may be mentioned anethole, 1,4-cineole, dibenzyl ether, linalool oxide, limonene oxide, nerol oxide, rose oxide, methylisoeugenol, methyl chavicol, isoamyl phenylethyl ether, β-naphthyl methyl ether, phenyl propyl ether, p-cresyl methyl ether, vanillyl butyl ether, α-terpinyl methyl ether, citronellyl ethyl ether, geranyl ethyl ether, rose-furan, theaspirane, decyl methyl ether, methyl phenylmethyl ether and the like.

As the lactones, for example, there may be mentioned γ- or δ-decalactone, γ-heptalactone, γ-nonalactone, γ- or δ-hexalactone, γ- or δ-octalactone, γ- or δ-undecalactone, δ-dodecalactone, δ-2-decenolactone, methyllactone, 5-hydroxy-8-undecenenoic acid δ-lactone, jasmine lactone, menthalactone, dihydrocoumarin, octahydrocoumarin, 6-methylcoumarin and the like.

As the furans, for example, there may be mentioned furan, 2-methylfuran, 3-methylfuran, 2-ethylfuran, 2,5-diethyltetrahydrofuran, 3-hydroxy-2-methyltetrahydrofuran, 2-(methoxymethyl)furan, 2,3-dihydrofuran, furfural, 5-methylfurfural, 3-(2-furyl)-2-methyl-2-propenal, 5-(hydroxymethyl)furfural, 2,5-dimethyl-4-hydroxy-3(2H)-furanone (furaneol), 4,5-dimethyl-3-hydroxy-2(5H)furanone (sotolone), 2-ethyl-4-hydroxy-5-methyl-3(2H)-furanone (homofuranol), 5-ethyl-3-hydroxy-4-methyl-2(5H)furanone (homosotolone), 3-methyl-1,2-cyclopentanedione (cyclotene), 2(5H)-furanone, 4-methyl-2(5H)-furanone, 5-methyl-2(5H)-furanone, 2-methyl-3(2H)-furanone, 5-methyl-3(2H)-furanone, 2-acetylfuranone, 2-acetyl-5-methylfuran, furfuryl alcohol, methyl 2-furancarboxylate, ethyl 2-furancarboxylate, furfuryl acetate and the like.

As the hydrocarbons, for example, there may be mentioned α- or β-bisabolene, β-caryophyllene, p-cymene, terpinene, terpinolene, cadinene, farnesene, limonene, ocimene, myrcene, α- or β-pinene, 1,3,5-undecatriene, valencene and the like.

Moreover, as the acids, for example, there may be mentioned geranic acid, dodecanoic acid, myristic acid, stearic acid, lactic acid, phenylacetic acid, pyruvic acid, trans-2-methyl-2-pentenoic acid, 2-methyl-cis-3-pentenoic acid, 2-methyl-4-pentenoic acid, cyclohexanecarboxylic acid and the like.

Furthermore, as the natural origin aromatic materials, for example, there may be mentioned anise, orange, lemon, lime, mandarin, petit grain, bergamot, lemon balm, grapefruit, elemi, olibanum, lemon grass, neroli, marjoram, angelica root, star anise, basil, bay, calamus, chamomile, caraway, cardamom, cassia, cinnamon, pepper, perilla, cypress, oregano, cascarilla, ginger, parsley, pine needle, sage, hyssop, tea tree, mustard, horseradish, clarisage, clove, cognac, coriander, estragon, eucalyptus, fennel, guaiac wood, dill, cajuput, worm seed, pimento, juniper, fenugreek, garlic, laurel, mace, mil, nutmeg, spruce, geranium, citronella, lavender, lavandin, palmarosa, rose, rosemary, sandalwood, oak moth, cider wood, vetiver, linaloe, bois de rose, patchouli, labdanum, cumin, thyme, ylang-ylang, birth, capsicum, celery, tolu balsam, djenne, inmortel, benzoin, jasmine, cassia, tuberose, mignonette, marigold, mimosa, opopanax, orris, vanilla, licorice and the like. The flavor components contained in these natural origin aromatic materials can be also used.

According to the invention, an optional component to be blended in an antioxidant can be incorporated into the mixture A or mixture B within such a range that the advantages of the invention are not impaired. As the optional component usable, for example, a surfactant, a pigment, an antibacterial agent, an antiseptic, a fragrance-retaining agent, a freeze-stabilizer, a deodorant base, an essential oil, a solvent and the like can be exemplified.

The physical object wherein its antioxidation function is desired to be exhibited using the double preparation type antioxidant of the invention is not particularly limited. For example, fragrances or cosmetics, oral products and the like can be exemplified, though not limited thereto.

As the perfumes or cosmetics, for example, perfume, Eau de Toilette, Eau de Cologne, shower cologne and the like fragrance products, skin cream, cleansing cream, cleansing foam, skin lotion, aftershave lotion, foundation, lipstick, talcum powder and the like foundation cosmetics, shampoo, rinse, conditioner, rinse in shampoo, treatment, pomade, hair tonic, hair liquid, hair jell, revitalizing hair tonic, hair dye, cold wave and the like hair cosmetics, bath agent, clothing detergent, soft finish agent, kitchen detergent, toilet detergent, bath room detergent, glass cleaner, mildew remover and the like detergents, and the like can be exemplified.

The amount of the double preparation type antioxidant to be added to and blended with the physical object cannot be categorically defined because it sharply varies depending on the physical object, but in general, it is desirable to set it to from 0.0000001% by weight to 50% by weight based on the physical object and it is more desirable to set it to from 0.00001% by weight to 10% by weight.

There is no particular limitation regarding the method for exerting the antioxidation function using the double preparation type antioxidant of the invention. For example, it is desirable that the mixture A and mixture B are mixed at almost the same time and then applied to a site where the antioxidation function is desired to be exhibited, but either one of the mixture A and mixture B may be applied to the site where the antioxidation function is desired to be exhibited and then the other one may be applied to the site where the antioxidation function is desired to be exhibited, thereby effecting their mixing. By the use of the double preparation type antioxidant of the invention, influences by an active oxygen and/or a free radical or the like can be avoided or reduced.

In this connection, according to the invention, ratio of the amount of the mixture A to be used and the amount of the mixture B to be used is not particularly limited. This sharply varies depending on the kinds and amounts of the effective components contained in the respective components to be used, conditions of the physical object to be subjected to antioxidation, and the like. In this connection, it is desirable that pH of a mixture of the mixture A and mixture B is set to 7 to 11.

According to the invention, the mixture A and mixture B can be separately stored to form an antioxidation product. The separately storing means is not particularly limited, but as a preferred means, for example, there may be mentioned an antioxidant component-storing container which comprises containers in which the mixture A and mixture B are separately stored and wherein at least part of these containers are mutually combined. It is preferable that the two containers are prepared by molding as an integrated container.

Illustratively, there may be exemplified a storing container wherein the mixture A and mixture B are stored in liquid states of the individual two liquids without mixing in a container comprising individual containers, e.g., a storing container having two storing parts storing the two liquids with a partition member in one container, in which the above-mentioned individual components are stored, or a storing container wherein the above-mentioned individual component liquids are stored in two independent containers and the independent containers are integrally combined with each other by a fixing mechanism or detachable mechanism such as fitting, joining or adhesion, or further an integrated storing container wherein two independent type containers are set in a tray.

As a particularly preferred storing container, there may be mentioned a tubular container, which comprises a thin plate-type partition member or a tubular partition member having almost the same shape as that of the aforementioned container as the partition member in the container. The storing container is partitioned by said partition member to form two storing parts and they are separately stored in respective storing parts. When an antioxidation function is desired to be exhibited, the mixture A and mixture B are extruded from the respective outlets, mutually mixed around the outlets (a site of the outlets in the extruding direction), and applied to a physical object wherein its antioxidation function is desired to be exhibited. As an illustrative example, a toothpaste tube can be exemplified but is not limited thereto in the invention.

Moreover, as a preferred container different from the above, there is a spray-type container. The container is classified into a gas-type one and a mist-type one, and all these types of containers are included in the invention. In order to avoid complexity in explanation and difficulty in understanding, the explanation of the aforementioned types is omitted in the following.

A spray-type container comprising two liquid-storing parts inside the container is also one of preferred storing containers of the invention, wherein the storing container comprises a hollow tube stored in the respective liquid-storing parts and immersed in the stored components at one end, the aforementioned components are mutually combined in the vicinity of another end of said hollow tube, and said another end is connected to a spraying part. The mixture A and mixture B are stored in respective two storing parts of the storing container. When an antioxidation function is desired to be exhibited, the mixture A and mixture B are passed through respective hollow tubes, mutually mixed at a combined part of the hollow tubes, passed through an end part (outlet) of the hollow tube, and passed through the spraying part to spray to the physical object in which the antioxidation function is to be exhibited.

In this connection, according to the invention, an agent I containing the mixture A may be stored in one of the aforementioned two containers, and an agent II containing the mixture B may be stored in the other. Also, according to the invention, it is possible also to make a fragrance or cosmetic, oral product or the like into a double preparation type one by dividing it into two, using one of them as the agent I by allowing it to contain a mixture A comprising a polyphenol and using the other as the agent II by allowing it to contain a mixture B comprising an alkali agent. The basis, method and the like for dividing into two are not particularly limited.

In addition, another embodiment of the invention is as follows.

(1) An antioxidation method or active oxygen elimination method, wherein a double preparation type antioxidant comprising a mixture A comprising a polyphenol and a mixture B comprising an alkali agent and an aqueous solvent is applied to an active oxygen and/or a free radical.

(2) An antioxidation method or active oxygen elimination method, wherein a mixture A comprising a polyphenol and a mixture B comprising an alkali agent and an aqueous solvent is successively applied to an active oxygen and/or a free radical.

### EXAMPLES

The following illustratively describes the invention by way of examples and the like, but the invention is by no means limited to the following examples.

### Reference Example 1

### Preparation of Coffee Raw Bean Extract

After grinding 1 kg of raw coffee beans (mesh 5 mm), 6 liters of water was added thereto to carry out extraction at from 85 to 95° C. for 2 hours. After filtering the total amount of this extract, the filtrate was allowed to be adsorbed by a column of XAD-2 (manufactured by Organo Corporation). After washing with water and subsequent elution with methanol, the result was concentrated and dried to obtain 52 g of a coffee raw bean extract containing polyphenol.

### Reference Example 2

### Preparation of Tea Extract

By extracting 1 kg of natural leaf tea (green tea) while stirring for 1 hour in 10 liters of hot water of 90° C. and removing the tea leaves by filtration, 8.3 liters of an extract was obtained. This liquid was concentrated to 1 liter, and 1 liter of acetone was added and it was stirred, and the thus formed insoluble matter was removed by centrifugation. 1 liter of ethyl acetate was added to the supernatant liquid and stirred, followed by 30 minutes of standing. The thus obtained ethyl acetate layer was concentrated under a reduced pressure, converted into water layer and then freeze-dried to obtain 97 g of tea extract containing a tea polyphenol having a purity of 60%.

### Example 1

### Preparation of Double Preparation Type Antioxidant

A mixture A (pH=4.6) was obtained by diluting 0.1 g of the coffee raw bean extract of Reference Example 1 with 100 ml of purified water. A mixture B (pH is 9.0) comprising sodium hydrogen carbonate in a concentration of 0.1% was obtained.

### Test Example 1

### Radical Elimination Effect of the Double Preparation Type Antioxidant of Example 1

The mixture A and mixture B of Example 1 were added in equivalent amount and mixed at room temperature to prepare a double preparation type antioxidant. The double preparation type antioxidant (sample) was diluted with purified water to have the polyphenol concentrations shown in Table 1 and maintained for the periods shown in Table 1, and then its radical elimination effect was verified by the method described in the following.

### (Verification Method of Radical Elimination Effect)

By allowing DPPH (1,1-diphenyl-2-picrylhydrazy) which is one kind of free radicals to react with the double preparation type bath agent of Example 1, the radical elimination reaction in which the purple DPPH ethanol solution is decolorized was evaluated by the following test.

### (Operation)

A sample was potted on No. 2 filter paper using a glass capillary. The spot was naturally dried. The radical elimination reaction was verified by spraying a 1 mM DPPH ethanol solution on the filter paper using a sprayer and confirming that the purple color was discolored. The verification results are based on the observation by 5 panelists with visual contact and the conclusion of half or more of the panelists.

Results of verification of the radical elimination reaction are shown in Table 1.

### (Preparation of 1 mM DPPH Ethanol Solution)

A 0.019716 g of DPPH was precisely weighed and added to a volumetric flask. This was filled up to 50 ml by poring ethanol (prepare when used).

TABLE 1

| Polyphenol | Double preparation | | | | | | |
|---|---|---|---|---|---|---|---|
| concentration | type antioxidant | | | Control | | | |
| (PPM) | 0 hr | 2 hrs | 24 hrs | 0 hr | 2 hrs | 24 hrs | pH |
| 200 | + | + | + | + | + | + | 9.3 |
| 150 | + | + | + | + | + | + | 9.3 |
| 100 | + | + | + | + | ± | ± | 9.3 |
| 80 | + | + | + | − | − | − | 9.3 |
| 60 | + | + | + | − | − | − | 9.4 |
| 40 | ± | ± | − | − | − | − | 9.4 |

In the table, the+shows that the DPPH radical elimination ability is positive, the±shows that the DPPH radical elimination ability is quasi positive, and the−shows that the DPPH radical elimination ability is negative (the same shall apply hereinafter).

In addition, the pH is a pH of the double preparation type antioxidant (the same shall apply hereinafter).

Purified water was used in the control instead of the sodium hydrogen carbonate aqueous solution as the agent II which comprises the mixture B, but the polyphenol concentration was made even.

Based on the results of Table 1, it was able to be confirmed that the double preparation type antioxidant of the invention has the antioxidant ability even at a polyphenol concentration of 60 ppm. Since the control has the antioxidant ability at 150 ppm on the other hand, it was revealed that the double preparation type antioxidant of the invention is excellent in the antioxidative potential.

### Example 2

### Preparation of Double Preparation Type Bath Agent (Preparation of Agent I Which Comprises the Mixture A)

Predetermined amounts of the tea extract of Reference Example 2, dipropylene glycol (DPG) and purified water were weighed to have their concentrations of 2.0% by weight, 48.0% by weight and 50.0% by weight, respectively, and then these were mixed and dissolved by stirring at room temperature, thereby obtaining the agent I which comprises the mixture A. The pH was 5.8.

### (Preparation of Agent II Which Comprises the Mixture B)

Predetermined amounts of sodium hydrogen carbonate, DPG and purified water were weighed to have their concentrations of 4.0% by weight, 46.0% by weight and 50.0% by weight, respectively, and then these were mixed and dissolved by stirring at room temperature, thereby obtaining the agent II which comprises the mixture B. The pH was 8.9.

### (Preparation of Double Preparation Type Bath Agent)

A double preparation type bath agent was prepared by mixing the agent I which comprises the mixture A and the agent II which comprises the mixture B at 1:1 (weight) and at room temperature.

### Test Example 2

### Radical Elimination Effect of the Double Preparation Type Bath Agent of Example 1

The double preparation type bath agent (sample) of Example 2 was diluted with purified water such that it was contained as described in Table 2, and its radical elimination effect was verified by the same method described in the above. The results of verifying the radical elimination reaction are shown in Table 2.

TABLE 2

| Sample | Polyphenol | | |
|---|---|---|---|
| concentration | concentration | Double component | |
| (% by weight) | (RPM) | type bath agent | Control |
| 5.00 | 500 | + | + |
| 4.00 | 400 | + | + |
| 3.00 | 300 | + | + |
| 2.00 | 200 | + | + |
| 0.80 | 80 | + | − |
| 0.60 | 60 | + | − |
| 0.40 | 40 | + | − |
| 0.20 | 20 | + | − |

In the control, the mixture A alone was used and the polyphenol concentration was made even.

Based on the results of Table 2, it was able to be confirmed that the double preparation type bath agent of the invention has the antioxidant ability even at a polyphenol concentration of 20 ppm. Since the control has the antioxidant ability at 200 ppm on the other hand, it was found that the double preparation type bath agent of the invention is excellent in the antioxidative potential.

### Example 3

### Preparation of Double Preparation Type Cleansing Article (Preparation of Agent I Which Comprises the Mixture A)

An agent I which comprises the mixture A was prepared by mixing, at room temperature, a polyphenol powder GTO 90 (manufactured by Aiya Japan Corporation) and an weakly acidic cleansing article having the composition of Table 3 at the quantitative ratios described in Table 4. The pH was 5.6.

TABLE 3

| (Component names) | (% by weight) |
|---|---|
| N-Lauroyl-sodium L-glutamate | 20.00 |
| 1,3-Butylene glycol | 50.00 |
| Citric acid | 5.00 |
| Purified water | 25.00 |
| | 100.00 |

pH = 5.5

TABLE 4

| | Amount of polyphenol | Amount of weakly acidic | |
|---|---|---|---|
| | powder GTO | cleansing article | Total |
| No. | (% by weight) | (% by weight) | (% by weight) |
| 1 | 5.0 | 95.0 | 100.0 |
| 2 | 4.0 | 96.0 | 100.0 |
| 3 | 3.0 | 97.0 | 100.0 |
| 4 | 2.0 | 98.0 | 100.0 |
| 5 | 1.0 | 99.0 | 100.0 |
| 6 | 0.8 | 99.2 | 100.0 |
| 7 | 0.6 | 99.4 | 100.0 |
| 8 | 0.4 | 99.6 | 100.0 |
| 9 | 0.2 | 99.8 | 100.0 |

### (Preparation of Agent II Which Comprises the Mixture B)

An agent II which comprises a mixture B having the composition of Table 5 was prepared. The pH was 8.7.

TABLE 5

| (component names) | (% by weight) |
|---|---|
| Stearic acid | 16.00 |
| Palmitic acid | 7.00 |
| Myristic acid | 7.00 |
| Polyethylene glycol monostearate | 6.00 |
| Polyoxyethylene lauryl ether | 2.00 |
| d-δ-Tocopherol | 0.05 |
| concentrated glycerin | 10.00 |
| p-Hydroxybenzoic acid ester | 0.10 |
| Potassium hydroxide | 5.20 |
| Purified water | 16.00 |
| Diethanol amide laurate | 2.50 |
| Purified water | 28.05 |
| p-Hydroxybenzoic acid ester | 0.10 |
| | 100.00 |

pH = 8.7

### (Preparation of Double Preparation Type Cleansing Article)

A double preparation type cleansing article was prepared by mixing the agent I which comprises the mixture A and the agent II which comprises the mixture B at 1:1 (weight) and at room temperature.

### Test Example 3

### Radical Elimination Effect of the Double Preparation Type Cleansing Article of Example 3

The radical elimination effect was verified by the same method of Test Example 1 except that the double preparation type cleansing article (sample) of Example 3 was diluted with purified water to have 2% by weight. The results of verifying the radical elimination effect are shown in Table 6.

TABLE 6

| Sample | Polyphenol | | |
|---|---|---|---|
| concentration | concentration | Double component | |
| (% by weight) | (RPM) | type bath agent | Control |
| 5.00 | 500 | + | + |
| 4.00 | 400 | + | + |
| 3.00 | 300 | + | + |
| 2.00 | 200 | + | + |
| 0.80 | 80 | + | + |
| 0.60 | 60 | + | − |
| 0.40 | 40 | + | − |
| 0.20 | 20 | + | − |

In the control, the mixture A alone was used and the polyphenol concentration was made even.

Based on the results of Table 4, it was able to be confirmed that the double preparation type cleansing article of the invention has the antioxidant ability even at a polyphenol concentration of 20 ppm. Since the control has the antioxidant ability at 200 ppm on the other hand, it was found that the double preparation type cleansing article of the invention is excellent in the antioxidative potential.

### (Preparation Example)

A bath agent in which the mixture A and mixture B are the following mixtures, respectively, can be exemplified as a preferable double preparation type bath agent of the invention, but the invention is not limited to this bath agent.

TABLE 7
Mixture A

| (Component name) | (% by weigh) |
|---|---|
| Gallic acid | 2.00 |
| Neutral anhydrous sodium sulfate | 97.50 |
| Silicic acid anhydride | 0.50 |
| | 100.00 |

Mixture B

| (Component name) | (% by weight) |
|---|---|
| Sodium bicarbonate | 99.50 |
| Silicic acid anhydride | 0.50 |
| | 100.00 |

While the invention has been described in detail and with reference to specific embodiments thereof, it will be apparent to one skilled in the art that various changes and modifications can be made therein without departing from the spirit and scope of the invention.

In this connection, this application is based on a Japanese patent application filed on Dec. 22, 2006 (Japanese Patent Application No. 2006-346658), the entire contents thereof being thereby incorporated by reference.

1. A double preparation type antioxidant, which comprises a mixture A comprising a polyphenol and a mixture B comprising an alkali agent.

2. The double preparation type antioxidant according to claim 1, wherein the mixture B further comprises an aqueous solvent.

3. The double preparation type antioxidant according to claim 1, wherein an amount of the polyphenol contained in the mixture A is from 10⁻⁷ to 10% by mass.

4. The double preparation type antioxidant according to claim 1, wherein pH of an admixture of the mixture A and mixture B is from 7 to 11.

5. A double preparation type antioxidation product, which comprises two antioxidant component-storing containers wherein at least parts of the two antioxidant component-storing containers are mutually integrally molded and combined,
wherein a mixture A comprising a polyphenol is stored in one of the antioxidant component-storing containers, and a mixture B comprising an alkali agent is stored in the other antioxidant component-storing container.

6. The double preparation type antioxidation product according to claim 5, which further comprises a structure in which the two antioxidant component-storing containers each provide outlets, wherein the mixture A and the mixture B are mutually mixed when emitted through their respective outlets of the two antioxidant component-storing containers.

7. A double preparation type fragrance or cosmetic, which comprises an agent I comprising a mixture A comprising a polyphenol and an agent II comprising a mixture B comprising an alkali agent.

8. A double preparation type oral product, which comprises an agent I comprising a mixture A comprising a polyphenol and an agent II comprising a mixture B comprising an alkali agent.
